(19) United States
(12) Patent Application Publication
(10) Pub. No.: US 2015/0305598 A1
(43) Pub. Date: Oct. 29, 2015

(54) ENDOSCOPE

(71) Applicant: OLYMPUS CORPORATION, Tokyo (JP)

(72) Inventor: Toshihiro YAMASHITA, Tokyo (JP)

(73) Assignee: OLYMPUS CORPORATION, Tokyo (JP)

(21) Appl. No.: 14/794,051

(22) Filed: Jul. 8, 2015

Related U.S. Application Data
(63) Continuation of application No. PCT/JP2014/062350, filed on May 8, 2014.

(30) Foreign Application Priority Data
Aug. 30, 2013 (JP) 2013-180116

(51) Int. Cl. A61B 1/005

(52) U.S. Cl. CPC A61B 1/0055; A61B 1/0052

(57) ABSTRACT

An endoscope including a first bending portion that actively performs a bending action in conjunction with a bending operation in an operation portion, a second bending portion that is provided on a proximal end side of the first bending portion, and passively performs a bending action by an external force, a flexible tube portion that is consecutively connected to a proximal end side of the second bending portion, wherein a series of helical tube portions (a first flex and a second flex) are disposed in the second bending portion and in the flexible tube portion, the helical tube portion being configured such that bending rigidity at a distal end side is higher than bending rigidity at a proximal end side, a boundary at which the bending rigidity of the helical tube portions change (a boundary between the first flex and the second flex) is set to a distal end side of the flexible tube portion, to thereby prevent buckling of the bending portion and realize good insertability of an insertion portion.

### CROSS REFERENCE TO RELATED APPLICATION

This application is a continuation application of PCT/JP2014/062350 filed on May 8, 2014 and claims benefit of Japanese Application No. 2013-180116 filed in Japan on Aug. 30, 2013, the entire contents of which are incorporated herein by this reference.

### BACKGROUND OF THE INVENTION

1. Field of the Invention

The present invention relates to an endoscope provided with a bending portion that passively bends by an external force, on a distal end side of a flexible tube portion.

2. Description of the Related Art

Conventionally, an endoscope in a medical field is used for observing a target region in a body by inserting an elongated insertion portion into the body, and for performing various types of treatments using a treatment instrument which is inserted into a treatment instrument insertion channel as necessary. In the insertion portion of the endoscope of this type, a distal end portion, a bending portion and a flexible tube portion are disposed in this order from a distal end side, and a surgeon or the like is allowed to bend the bending portion in a desired direction by performing a predetermined operation of an operation knob or the like disposed at an operation portion of the endoscope while grasping the flexible tube portion and pushing it into the body when the insertion portion is inserted into the body.

Incidentally, with respect to such an insertion portion of the endoscope, various contrivances have been provided in order to improve insertability into a bending interior of the body. For example, in Japanese Patent Laid-Open Publication No. 58-49132, there is disclosed a technique in which, on a proximal end side of a first bending portion having a plurality of bending pieces which actively perform a bending action in conjunction with an operation for bending at the operation portion, a second bending portion which passively performs a bending action in accordance with an external force is provided. Here, in the technique in Japanese Patent Laid-Open Publication No. 58-49132, the second bending portion is configured by extending a helical core material (flex) which constitutes the flexible tube portion to the bending portion, and integrally covering an outer circumferential surface of the extended core material and the bending pieces of the first bending portion by an elastic tube which is more flexible than an outer fitting tube fitted on and covering the flexible tube portion.

### SUMMARY OF THE INVENTION

An endoscope according to an aspect of the present invention includes: an elongated insertion portion; an operation portion that is consecutively connected to a proximal end side of the insertion portion and allows to perform a bending operation; a first bending portion provided in the insertion portion and including a bending structure that actively performs a bending action in conjunction with the bending operation; a second bending portion that is provided in the insertion portion and consecutively connected to a proximal end side of the first bending portion, and that passively performs a bending action by an external force; a flexible tube portion that has flexibility, and is provided in the insertion portion and consecutively connected to a proximal end side of the second bending portion; a helical tube portion that is disposed in the second bending portion and in the flexible tube portion, the helical tube portion being formed by helically winding a belt-shaped member such that bending rigidity at a distal end side is higher than bending rigidity at a proximal end side; a first outer cover that covers outer circumferential sides of the bending structure in the first bending portion and the helical tube portion in the second bending portion; a second outer cover that covers an outer circumferential side of the helical tube portion in the flexible tube portion and has hardness greater than hardness of the first outer cover; and a boundary portion that is provided at a distal end side of the flexible tube portion and at which the bending rigidity of the helical tube portion changes.

### BRIEF DESCRIPTION OF THE DRAWINGS

FIG. 1 is a perspective view showing a configuration of an endoscope according to a first embodiment of the present invention;

FIG. 2 is an explanatory view of a distal end part of an insertion portion according to the first embodiment;

FIG. 3 is a side sectional view of principal parts showing a second bending portion and a flexible tube portion in a state where a first outer cover is removed, according to the first embodiment;

FIG. 4 is an exploded perspective view showing principal parts of a bending portion and the flexible tube portion;

FIG. 5 is an explanatory view showing a state where the insertion portion is inserted into an upper lobe of bronchia according to the first embodiment;

FIG. 6 is a side sectional view of principal parts showing a second bending portion and a flexible tube portion in a state where a first outer cover is removed, according to a second embodiment of the present invention;

FIG. 7 is a perspective view of a flex according to the second embodiment of the present invention;

FIG. 8 a side sectional view of principal parts showing a second bending portion and a flexible tube portion in a state where a first external cover is removed, according to a third embodiment of the present invention;

FIG. 9 is an exploded perspective view showing principal parts of a bending portion and the flexible tube portion according to the third embodiment of the present invention; and

FIG. 10 is an enlarged sectional view showing a modified example of the flex according to the third embodiment of the present invention.

### DETAILED DESCRIPTION OF THE PREFERRED EMBODIMENTS

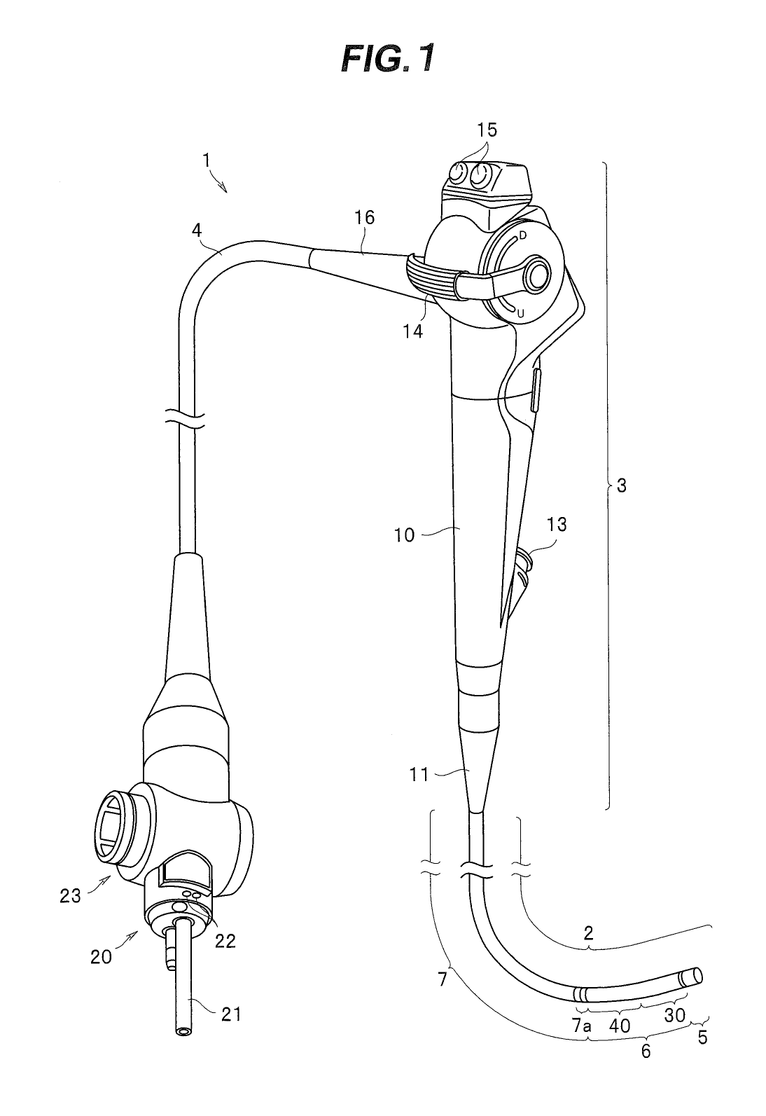

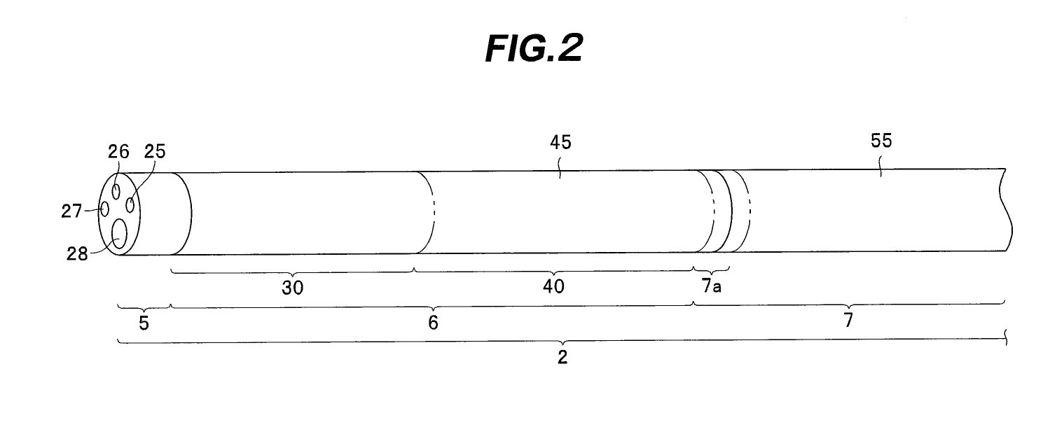

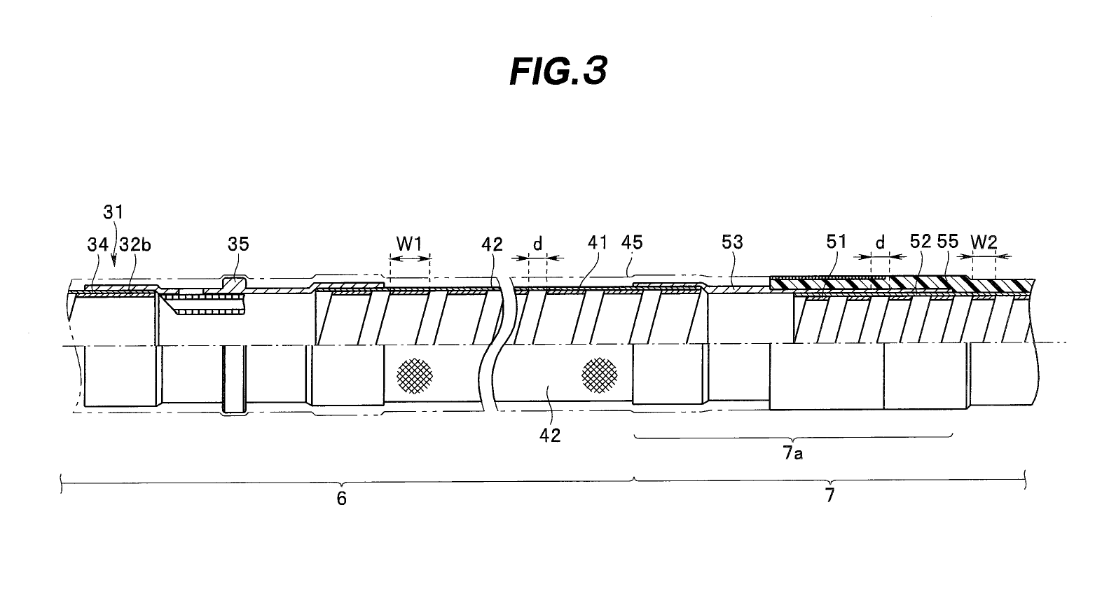

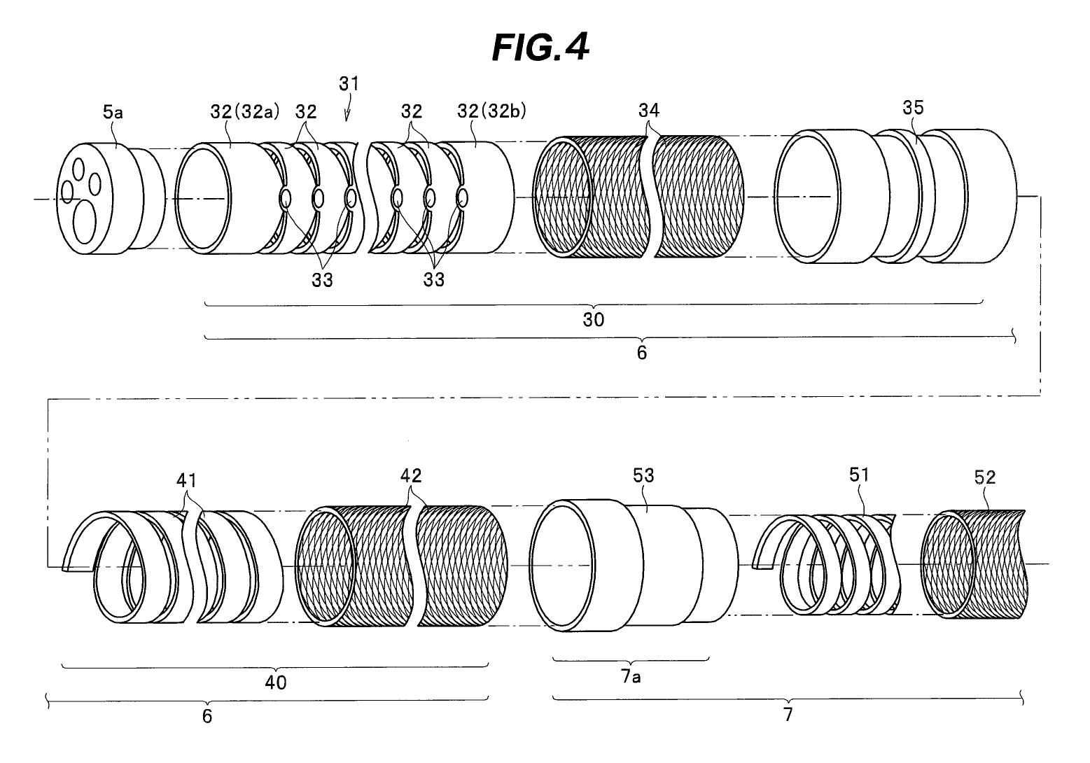

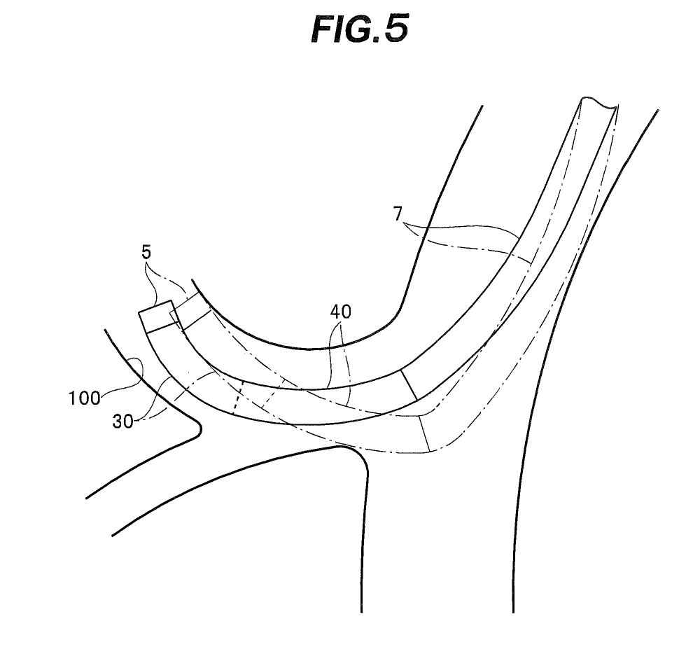

Hereinafter, aspects of the present invention will be described referring to the drawings. FIGS. 1 through 5 relate to a first embodiment of the present invention, and FIG. 1 is a perspective view showing a configuration of an endoscope, FIG. 2 is an explanatory view of a distal end part of an insertion portion, FIG. 3 is a side sectional view of principal parts showing a second bending portion and a flexible tube portion in a state where a first outer cover is removed, FIG. 4 is an exploded perspective view showing principal parts of a bending portion and the flexible tube portion, and FIG. 5 is an explanatory view showing a state where the insertion portion is inserted into an upper lobe of bronchia.

An endoscope 1 shown in FIG. 1 is, for example, an endoscope for bronchia (a bronchoscope). The endoscope 1 is configured to include an insertion portion 2 which is elongated and insertable into a target region such as bronchia in a subject, an operation portion 3 consecutively connected to a proximal end side of the insertion portion 2, a universal cord 4 extended from a side portion of the operation portion 3.

The operation portion 3 includes an operation portion body 10 which constitutes an operation grasping portion, and a distal end side of the operation portion body 10 is connected to a proximal end side of the insertion portion 2 through a bending prevention portion 11. Further, at a location close to a distal end of the operation portion body 10, there is provided a treatment instrument insertion port 13 as an opening on a proximal end side of a treatment instrument insertion channel 28 (as descried later) which is a conduit for insertion of a treatment instrument into the insertion portion 2. On the other hand, at a location close to a proximal end of the operation portion body 10, an angle lever 14 is provided and switches 15 for various endoscope functions are provided.

One end side of the universal cord 4 is consecutively connected to a side portion of the operation portion body 10 through a bending prevention portion 16. On the other hand, at an extended end which is the other end side of the universal cord 4, a scope connector portion 20 is provided. At an end portion of the scope connector portion 20, there is provided a light source side connector 21 attachable to and detachable from a light source apparatus that is not shown. At the light source side connector 21, a proximal end portion of a light guide (not shown) extending from a side of the insertion portion 2 is provided to protrude, and electric contacts 22 are disposed, and when the light source side connector 21 is connected to the light source apparatus, the light guide is optically connected with a light source in the light source apparatus and the electric contacts 22 are electrically connected with a power supply in the light source apparatus. Further, at a side portion of the scope connector portion 20, there is provided an electric connector 23 attachable to and detachable from a video processor that is not shown.

As shown in FIGS. 2 through 4, the insertion portion 2 is configured with a distal end portion 5, a bending portion 6 and a flexible tube portion 7 consecutively connected in this order from a distal end, the bending portion 6 being bendable and disposed on a proximal end side of the distal end portion 5, the flexible tube portion 7 being elongated, having flexibility and disposed on a proximal end side of the bending portion 6.

For example, as shown in FIG. 2, in the distal end portion 5, an illumination optical system 25 for illuminating an interior of the subject and an image pickup optical system 26 for picking up an image of the subject are provided, and an air/water feeding channel 27 for supplying a fluid toward a target region in the subject and the treatment instrument insertion channel 28 through which a treatment instrument such as a forceps is led out and so forth are formed.

The bending portion 6 is configured to include, for example, a first bending portion 30 which is actively bendable in two directions of upward and downward in conjunction with a bending operation through the angle lever 14 of the operation portion 3, and a second bending portion 40 which is disposed on a proximal end side of the first bending portion 30 and passively performs a bending action by an external force.

As shown in FIG. 4, the first bending portion 30 includes a bending structure 31 in which a plurality of bending pieces 32 made of metal are pivotally connected by pivot portions 33 such as rivets. An outer circumference of the bending structure 31 is covered, for example, by a braid 34 which is cylindrical and formed by weaving fine wires of metal such as stainless steel. Further, a proximal end side of a distal end portion body 5a, which is made of metal and constitutes the distal end portion 5, is connected to a bending piece 32a positioned at the distal end of the bending pieces 32 which constitute the bending structure 31. Further, a distal end side of a first mouthpiece 35 in a cylindrical shape is outwardly fitted on a bending piece 32b which is positioned at the proximal end of the bending pieces 32 constituting the bending structure 31, and the braid 34 which covers an outer circumference of the bending piece 32b, and fixed thereto by soldering or the like. It is noted that, as not shown in the figure, distal end sides of two bending operation wires which are connected to the angle lever 14 are fixed to an inner circumference of the bending piece 32a positioned at the distal end of the bending pieces 32, and the bending operation wires are pulled or slacken by an operation of the angle lever 14 and thereby the first bending portion 30 actively performs a bending action in upward and downward directions.

On the other hand, the second bending portion 40 includes a first flex 41, as a helical tube member constituting a helical tube portion, for example, comprised of a compression coil spring which is configured by helically winding a belt-shaped spring steel made of stainless steel or the like. An outer circumference of the first flex 41 is covered, for example, by a braid 42 which is cylindrical and formed by weaving fine wires of metal such as stainless steel. Further, as shown in FIG. 3, a proximal end side of the first mouthpiece 35 is outwardly fitted on distal end sides of the first flex 41 and the braid 42 covering the outer circumference of the first flex 41, and fixed thereto by soldering or the like. On the other hand, a distal end side of a second mouthpiece 53 made of metal and forming a rigid portion 7a at a distal end side of the flexible tube portion 7 is outwardly fitted on proximal end sides of the flex 41 and the braid 42 covering the outer circumference of the flex 41, and fixed thereto by soldering or the like.

Further, outer circumferences of the braids 34 and 42 in the first and second bending portions 30 and 40 are integrally covered, for example, by a first outer cover 45 which is tubular and made of soft rubber or the like. It is noted that a distal end side of the first outer cover 45 is adhered and fixed, for example, to an outer circumferential portion of the distal end portion body 5a liquid-tightly, and a proximal end side of the first outer cover 45 is adhered and fixed, for example, to an outer circumferential portion of the second mouthpiece 53.

The flexible tube portion 7 includes a second flex 51, as a helical tube member constituting a helical tube portion, for example, comprised of a compression coil spring which is configured by helically winding a belt-shaped spring steel made of stainless steel or the like. An outer circumference of the second flex 51 is covered, for example, by a braid 52 which is cylindrical and formed by weaving fine wires of metal such as stainless steel. Further, as shown in FIG. 3, a proximal end side of the second mouthpiece 53 is outwardly fitted on distal end sides of the second flex 51 and the braid 52 covering the outer circumference of the second flex 51, and fixed thereto by soldering or the like. On the other hand, a proximal end side of the second flex 51 is extended inside the bending prevention portion 11 (not shown in the figure).

Further, an outer circumference of the braid 52 in the flexible tube portion 7 is covered, for example, by a second outer cover 55 which is tubular and made of resin or the like having predetermined hardness. It is noted that a distal end side of the second outer cover 55 is adhered and fixed, for example, to an outer circumferential portion of the second mouthpiece 53 liquid-tightly, and a proximal end side of the second outer cover 55 is extended inside of the bending prevention portion 11.

In the insertion portion 2 of the endoscope 1 as described above, the first outer cover 45 is configured with the soft rubber or the like, and thereby the first and second bending portions 30 and 40 are set as a region that is bendable. On the other hand, the second outer cover 55 is configured with the resin or the like having predetermined hardness, and thereby the flexible tube portion 7 does not bend easily as the bending portions 30 and 40 but is set as a region capable of performing a predetermined flexural deformation.

Further, in order to prevent a sharp change in rigidity between the bending portion 6 (the second bending portion 40) and the flexible tube portion 7 by a difference in hardness between the first outer cover 45 and the second outer cover 55, the first flex 41 is configured with a member having higher rigidity than the second flex 51 (a member having a higher spring constant K than the second flex 51).

Here, spring constants K of the flex 41 and 51 can be obtained according to the following equation.

K=G·d⁴/8Na·D³ (1)

It is noted that G is a spring coefficient, d is a board thickness, and D is an inner diameter in the equation (1).

As is clear from the equation (1), when an interval s of winding is set to be fixed, it is possible to make the spring constant K of the flex higher by increasing a board width W of the flex so that the effective number of turns Na is reduced.

Thus, as shown in FIGS. 3 and 4, the first flex 41 in the present embodiment is contemplated to have high rigidity by setting a flex board width W1 of the first flex 41 to be greater than a flex board width W2 of the second flex 51. Further, as described above, the proximal end side of the first flex 41 is connected to the second mouthpiece 53 provided at the rigid portion 7a of the flexible tube portion 7 (that is, a boundary between the first flex 41 and the second flex 51 is set at the distal end side of the flexible tube portion 7), and thereby it is prevented that a region having rigidity extremely lower than the flexible tube portion 7 is formed in the second bending portion 40.

According to the above embodiment, in the endoscope 1 including, in the insertion portion 2, the first bending portion 30 that actively performs a bending action in conjunction with a bending operation in the operation portion, the second bending portion 40 that is disposed on the proximal end side of the first bending portion and passively performs a bending action by an external force, and the flexible tube portion 7 that is consecutively connected to a proximal end side of the second bending portion 40, a series of helical tube portions (the first and second flexes 41 and 51) configured such that the bending rigidity at the distal end side is higher than the bending rigidity at the proximal end side is disposed in the second bending portion 40 and in the flexible tube portion 7, and the boundary at which the bending rigidity of the helical tube portions changes (the boundary between the first and second flexes 41 and 51) is set to the distal end side of the flexible tube portion 7, and thereby buckling of the second bending portion 40 is prevented and good insertability of the insertion portion 2 can be realized.

That is, the first flex 41 is configured with the helical tube member having higher rigidity than the second flex 51, and the boundary between these flexes is set to the distal end side of the flexible tube portion 7 and thereby a rigidity difference between the second bending portion 40 and the flexible tube portion 7 is set to be small without deteriorating operability in bending the first bending portion 30 (while maintaining flexibility of the first outer cove 45), and it can be prevented that a region where the rigidity is extremely lower than the flexible tube portion 7 is formed on the second bending portion 40. Therefore, for example, in a case of inserting the insertion portion 2 into an upper lobe of bronchia, as shown in FIG. 5, when the surgeon or the like grasps the flexible tube portion 7 and pushes the flexible tube portion into the body, buckling of the insertion portion 2 can be securely prevented from occurring in the vicinity of a boundary part between the flexible tube portion 7 and the second bending portion 40 (see the dashed line in FIG. 5).

In this case, particularly, since the helical tube portion is configured by connecting the two types of the helical tube members that are different in bending rigidity (the first and second flexes 41 and 51), and rigidities of the first and second flexes 41 and 51 can be set arbitrarily, it is possible to easily realize optimization of the bending rigidities of the second bending portion 40 and the flexible tube portion 7.

Further, the first flex 41 and the second flex 51 are connected with each other at the rigid portion 7a formed at the distal end side of the flexible tube portion 7, and thereby it can be prevented that a region where the rigidity changes due to the difference of the bending rigidities of the first flex 41 and the second flex 51 is newly formed on the flexible tube portion 7.

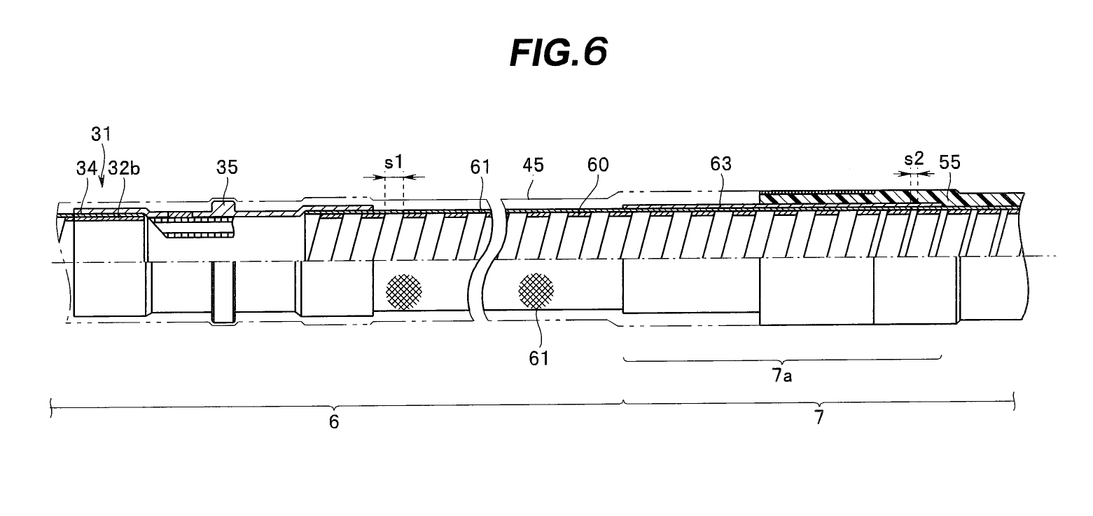

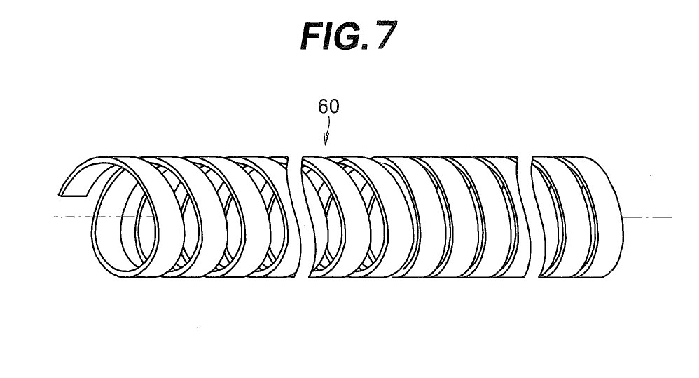

Next, FIGS. 6 and 7 relate to a second embodiment of the present invention, and FIG. 6 is a side sectional view of principal parts showing a second bending portion and a flexible tube portion in a state where the first outer cover is removed, and FIG. 7 is a perspective view of a flex. It is noted that the present embodiment differs from the above-described first embodiment mainly in the configuration of the helical tube portion. Regarding the other configurations which are the same as in the first embodiment, the same reference sings are assigned thereto and the description thereof is omitted.

As shown in FIGS. 6 and 7, in the insertion portion 2 according to the present embodiment, there is provided a flex 60, as a helical tube portion, which continues from a distal end side of the second bending portion 40 to a proximal end side of the flexible tube portion 7, and is comprised of one compression coil spring having a constant flex board width. The flex 60 is configured by helically winding a spring steel having a belt shape and made of stainless steel or the like. Here, in the flex 60 according to the present embodiment, an interval s1 of winding of the spring steel in a region at the distal end side is larger (to be coarser) than an interval s2 of winding of the spring steel at the proximal end side. Thus, by making winding density of the spring steel different and by forming the winding at the distal end side coarsely so that the effective number of turns Na in the equation (1) is made small, the flex 60s is configured such that rigidity (spring constant) in a region at the distal end side is set to be relatively higher than the rigidity in a region at the proximal end side.

An outer circumference of the flex 60 is covered, for example, by a braid 61 which is cylindrical and formed by weaving fine wires of metal such as stainless steel. Further, as shown in FIG. 6, the proximal end side of the first mouthpiece 35 is outwardly fitted on distal end sides of the flex 60 and the braid 61 covering the outer circumference of the flex, and fixed thereto by soldering or the like. On the other hand, a proximal end side of the flex 60 is extended inside the bending prevention portion 11 (not shown in the figure). Further, a boundary at which the rigidity changes in the flex 60 is set to the distal end side of the flexible tube portion 7. At the boundary, a second mouthpiece 63 made of metal and forming the rigid portion 7a at the distal end side of the flexible tube portion 7 is outwardly fitted on the flex 60 and the braid 61 covering the outer circumference of the flex, and fixed thereto by soldering or the like.

According to the above embodiment, in addition to the operational effects obtained in the foregoing first embodiment, an advantageous effect of simplifying the structure by disposing the flex 60, which is continuous, in the second bending portion 40 and the flexible tube portion 7.

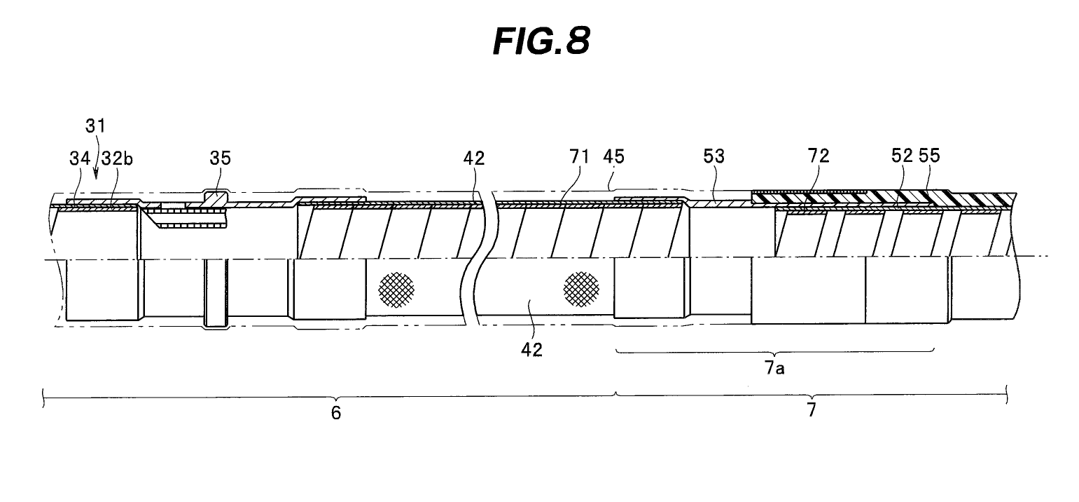

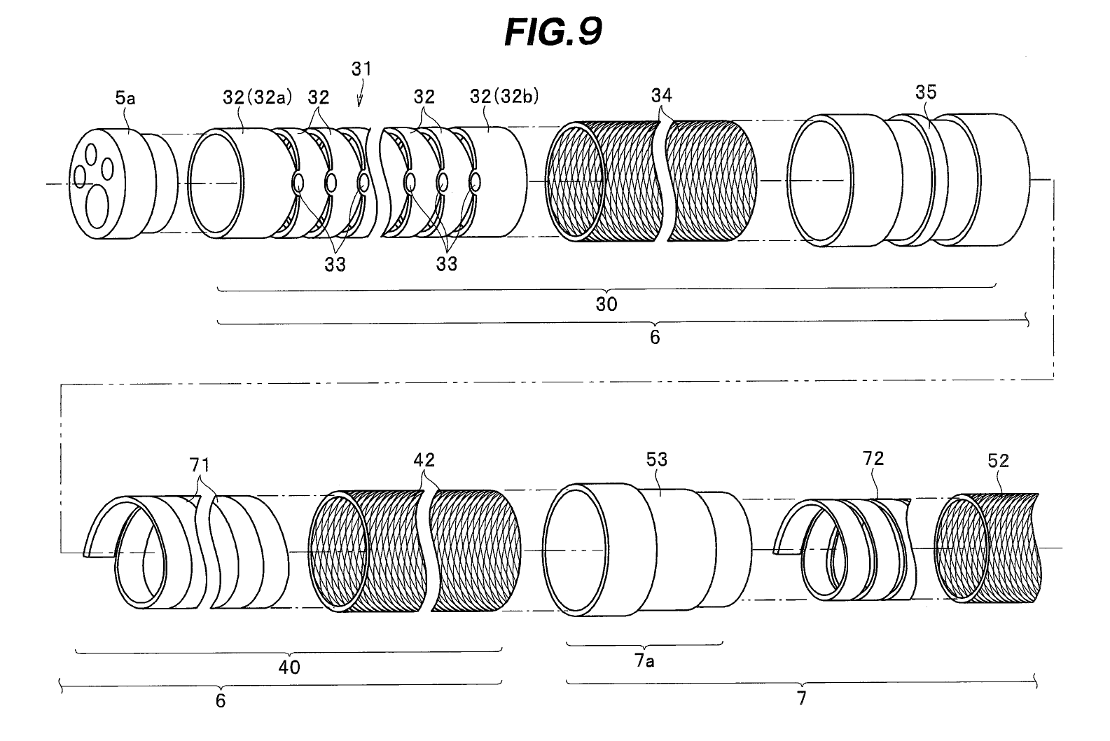

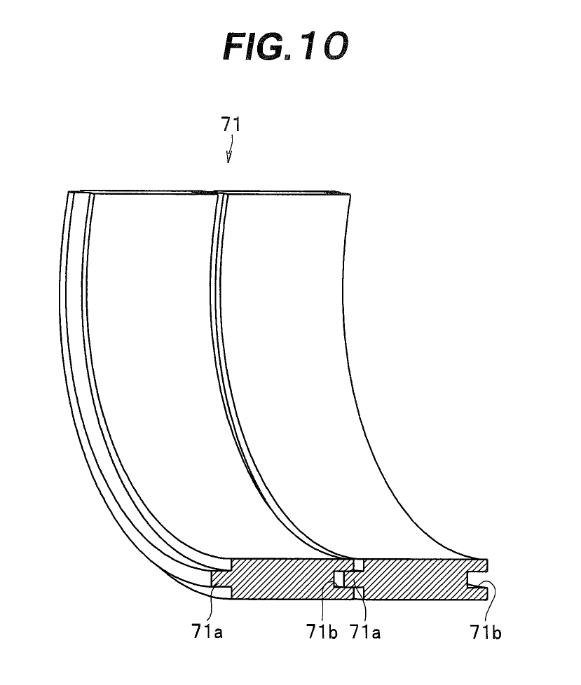

Next, FIGS. 8 through 10 relate to a third embodiment of the present invention, and FIG. 8 is a side sectional view of principal parts showing a second bending portion and a flexible tube portion in a state where the first outer cover is removed, FIG. 9 is an exploded perspective view showing principal parts of a bending portion and the flexible tube portion, and FIG. 10 is an enlarged sectional view showing a modified example of the flex. It is noted that the present embodiment differs from the above-described first embodiment mainly in the configuration of the helical tube portion. Regarding the others configurations which are the same as in the first embodiment, the same reference sings are assigned thereto and the description thereof is omitted.

As shown in FIGS. 8 and 9, in the present embodiment, the second bending portion 40 includes a first flex 71, as a helical tube member constituting a helical tube portion, comprised of a tension coil spring which is configured by helically winding a spring steel having a belt shape and made of stainless steel or the like.

Further, the flexible tube portion 7 includes a second flex 72, as a helical tube member constituting a helical tube portion, comprised of a compression coil spring which is configured by helically winding a spring steel having a belt shape and made of stainless steel or the like.

That is, in the present embodiment, the first flex 71 is configured with the tension coil spring in which an interval of winding of the spring steel is set to be substantially zero, and on the other hand, the second flex 72 is configured with the compression coil spring in which the spring steel is wound with a predetermined interval. Besides, the first and second flexes 71 and 72 can be configured with a continuous spring steel in the same manner as in the above-described second embodiment.

Here, differently from the spring constant K of the compression coil spring shown in the foregoing equation (1), spring constant K of the tension coil spring is expressed to further include a term of an initial tension T (an upper limit tensile force for maintaining a state of zero elongation), as shown in the following equation (2).

K=G·d⁴/8Na·D³+T (2)

Therefore, in the tension coil spring, the effective number of turns Na tends to be larger than that in the compression coil spring, but it is possible to set the spring constant K to be large in comparison with the compression coil spring by influence of the initial tension T.

Thus, in the present embodiment, by tuning the effective numbers of turns Na of the first and second flexes 71 and 72 taking the initial tension T into consideration, the spring constant of the first flex 71 is set to be larger than the spring constant of the second flex 72.

According to the above embodiment, substantially the same advantageous effect as obtained in the foregoing first embodiment can be obtained.

Here, as shown in FIG. 10, for example, a projecting stripe 71a is formed on one side surface (e.g. a side surface on the distal end side) of the spring steel which constitutes the first flex 71 and a recessed stripe 71b is formed on the other side surface (e.g. a side surface on the proximal end side), and it can be configured such that the projecting stripe 71a and the recessed stripe 71b are engaged with each other. With this configuration, it is possible to suppress the buckling of the second bending portion 40 more effectively.

Besides, the present invention is not limited to the above-described embodiments and various modifications and changes are possible, and those modifications and changes are within a technical scope of the present invention. For example, it is a matter of course that the configurations of the foregoing embodiments or modified examples may be appropriately combined.

Further, in the foregoing embodiments, one example in which the present invention is applied to the bronchoscope which particularly requires reduction in diameter is described, but the present invention is not limited to this and is applicable to an endoscope or the like for a digestive organ, a circulatory organ, brain surgery, a urinary organ, a genital organ, for example.

What is claimed is:

1. An endoscope comprising:
an elongated insertion portion;
an operation portion that is consecutively connected to a proximal end side of the insertion portion and allows to perform a bending operation;
a first bending portion provided in the insertion portion and including a bending structure that actively performs a bending action in conjunction with the bending operation;
a second bending portion that is provided in the insertion portion and consecutively connected to a proximal end side of the first bending portion, and that passively performs a bending action by an external force;
a flexible tube portion that has flexibility, and is provided in the insertion portion and consecutively connected to a proximal end side of the second bending portion;
a helical tube portion that is disposed in the second bending portion and in the flexible tube portion, the helical tube portion being formed by helically winding a belt-shaped member such that bending rigidity at a distal end side is higher than bending rigidity at a proximal end side;
a first outer cover that covers outer circumferential sides of the bending structure in the first bending portion and the helical tube portion in the second bending portion;
a second outer cover that covers an outer circumferential side of the helical tube portion in the flexible tube portion and has hardness greater than hardness of the first outer cover; and
a boundary portion that is provided at a distal end side of the flexible tube portion and at which the bending rigidity of the helical tube portion changes.

2. The endoscope according to claim 1, wherein the helical tube portion is configured by connecting or continuing two types of helical tube members which are different in bending rigidity.

3. The endoscope according to claim 1, wherein an interval of winding of the belt-shaped member changes and thereby the bending rigidity at the distal end side is set to be higher than the bending rigidity at the proximal end side.

4. The endoscope according to claim 1, wherein the helical tube portion comprises a compression coil spring, and an interval of winding of the belt-shaped member at the distal end side is set to be greater than an interval of winding of the belt-shaped member at the proximal end side, and thereby the bending rigidity at the distal end side is set to be higher than the bending rigidity at the proximal end side.

5. The endoscope according to claim 1, wherein the helical tube portion comprises a tension coil spring at the distal end side and a compression coil spring at the proximal end side, and thereby the bending rigidity at the distal end side is set to be higher than the bending rigidity at the proximal end side.
